(19) United States
(12) Patent Application Publication
(10) Pub. No.: US 2021/0318281 A1
(43) Pub. Date: Oct. 14, 2021

(54) LEVEL MEASURING AND GAS DETECTION SYSTEM

(71) Applicant: VEGA GRIESHABER KG, Wolfach (DE)

(72) Inventors: Florian KRAEMER, Muehlenbach (DE); Patrick HEIZMANN, Oberwolfach (DE); Stefan ALLGAIER, Oberwolfach (DE); Marcel DIETERLE, Schiltach (DE)

(73) Assignee: VEGA GRIESHABER KG, Wolfach (DE)

(21) Appl. No.: 17/230,945

(22) Filed: Apr. 14, 2021

(30) Foreign Application Priority Data
Apr. 14, 2020 (EP) PCT/EP2020/060458

(51) Int. Cl. G01N 33/00; G01N 27/16; G01N 35/00

(52) U.S. Cl. CPC G01N 33/0014; G01N 2035/0091; G01N 35/00722; G01N 27/16

(57) ABSTRACT

A level measuring and gas detection system arranged to measure a level and to detect a gas and/or an odor, with computing circuitry, arranged to generate a message when the level sensor detects a preset level limit value and/or when the gas sensor detects a preset gas concentration limit value.

### CROSS-REFERENCE TO RELATED APPLICATIONS

This application claims the benefit of the filing date of International Patent Application No. PCT/EP2020/060458 filed on 14 Apr. 2020, the entire content of which is incorporated herein by reference.

### FIELD

The disclosure relates to process automation. In particular, the disclosure relates to a level measuring and gas detection system, a particular use of a level measuring and gas detection system, a particular use of a gas sensor, a method of controlling a level of a container, a program element and a computer readable medium.

### TECHNICAL BACKGROUND

Especially in the industrial environment, level sensors are used to measure, i.e. detect the fill levels of containers. If the fill level reaches a predefined limit, the fill level sensor can be programmed to send a corresponding warning signal so that the container can be filled or emptied again.

Applications for such level sensors are not only found in industry, especially for monitoring production processes, but also, for example, in waste disposal for monitoring the fill level of waste containers.

### SUMMARY

There may be a desire to provide an extended level measuring system which can fulfil additional tasks.

This desire may be met by the features of the independent patent claims. Further embodiments of the present disclosure result from the subclaims and the following description of embodiments.

A first aspect of the present disclosure relates to a combined level measuring and gas detection system arranged to measure, i.e. detect a level or/and a threshold level and to detect a gas and/or an odor. The system comprises a level sensor arranged to detect the level of a medium in a container of a measuring point, and a gas sensor arranged to detect a gas and/or an odor and/or an odor intensity. Furthermore, a computing unit is provided which is arranged to generate a message and/or to instruct a process when the gas sensor detects a preset gas concentration limit value and/or a predetermined odor. The level sensor may be, for example, a level radar sensor, or a point level sensor or pressure sensor. The term level sensor should be interpreted broadly.

The term gas sensor is to be understood equally broadly in the context of the present disclosure. For example, it may be an odor sensor or an electronic nose. It should also be noted that the system may comprise multiple gas sensors. Further, the gas sensor may be arranged to detect odors, odorants and/or fragrances. The detection of odors can be used, for example, for direct quality monitoring of the medium.

A process may be any kind of process, such as actions and/or procedures. For example, it can be a calibration, a cleaning, a fermentation process or a quality monitoring of a medium, a locking of a sensor housing in an explosive environment, an emptying of the container in case of a filling level and/or odor and/or a control of a switching output and/or a pump control. It is also conceivable that a documentation of process-relevant procedures can be carried out.

The term “instruct” in the context of instructing the process with the computing device is to be interpreted broadly in the context of the present disclosure. A triggering and/or a control may be understood by it. For example, exceeding or falling below the gas concentration limit and/or an odor intensity may trigger the control of the process.

The computing unit may also be used to send a message when the computing unit detects an anomaly and/or an outlier via a neural network, such as via machine learning. The message may be displayed on the gas sensor, on another sensor, in a cloud and/or on a sensor system. The message may also be displayed on external output units such as a warning light or an acoustic signal generator. Furthermore, the message can be forwarded to mobile devices, control systems or PLCs.

Alternatively or additionally, a warning message can be generated when the fill level sensor detects a preset fill level limit. For example, the first warning message is generated when a maximum or minimum fill level of the container is reached. For example, the second warning is generated when the gas sensor detects an undesirable gas, at a certain concentration.

It can be provided that the warning message and/or the message is forwarded to a user or to a central control center. It is also possible that a central control center, a server or a cloud is supplied with the measurement data of the gas sensor and/or the level sensor and then generates the message and/or the warning message.

According to an embodiment, the system is set up to capture biometric data of the user, and in particular to perform facial recognition. It is also possible to perform Bluetooth recognition of the users mobile terminal.

The Bluetooth recognition may recognize recurring persons on the basis of the Bluetooth ID of the mobile end device without this person connecting to the sensor. For example, persons detected in the danger zone can be alerted or reported in a control system.

For example, with the help of an odor or several detected odors at the measuring point, an improved evaluation of a detected level and a detected gas or odor can be made possible in an advantageous manner. The detected odor(s) can be used, for example, to monitor or control the process or to monitor the medium.

According to an embodiment, the gas sensor is also set up to determine an image of an odor environment of the measuring point and, if necessary, to document or store it (locally, in the cloud, control system, etc. . . . ).

According to an embodiment, the level detection and gas detection system can be calibrated based on the detected odor. The calibration can be done manually or automatically. The calibration of the system can, for example, be self-learning. In this case, an automatic detection of odors and/or an automatic evaluation based on existing empirical values or predefined “odor molecules” can take place.

The system may also be automatically set and/or optimized for the medium. For example, the system may be set to perform a predetermined action when, for example, foam formation is detected.

According to an embodiment, the odor intensity limit value is an odor intensity limit value of a permitted or prohibited odor. Alternatively or additionally, the odor intensity limit value is an odor range.

According to an embodiment, the gas sensor is equipped with nanowires made of tin dioxide. The gas sensor or odor sensor can be equipped with nanowires made of tin dioxide, which are irradiated with UV light during measurement, which can cause a reduction of the electrical resistance. Scent molecules present in the air can accumulate on the surface of the tin dioxide and cause a change in the electrical resistance, which in turn can enable the odor to be measured.

According to an embodiment, the gas sensor is integrated in a mobile operating device for the level sensor. The mobile operating device can be, for example, a smartphone, a tablet or a wearable.

According to a further embodiment, the gas sensor is integrated in the level sensor.

According to a further embodiment, the computing unit is designed as a central server or as a cloud.

According to a further embodiment, the computing unit or part of the computing unit is integrated in the level sensor or in the mobile operating device.

According to a further embodiment, the computing unit is set up to calculate the position of the source of the odor and/or the gas or the gas outlet by evaluating the measurement data detected by the gas sensor.

At this point, it should be noted that the level measuring and gas detection system can in particular have a plurality of level sensors and a plurality of gas sensors which are (directly or indirectly) coupled to each other in such a way that they can communicate with each other, so that the measurement data recorded by all sensors can be evaluated. In particular, this enables a relatively precise localization of the position of the source of the gas outlet, as gas concentration data can be recorded by a large number of gas sensors arranged in a distributed manner.

Here, information on wind direction and wind strength (through local measurement or online services) may also be included in the calculation.

If, for example, a ring-shaped or spherical dispersion of the gas is assumed, the position of the source of the gas leakage can be concluded from several gas concentration measurement data carried out at different locations and from the knowledge of the different measurement locations.

According to another embodiment, the process initiated by the computing unit is to change a route of a waste disposal vehicle when the gas sensor detects the preset gas concentration limit.

In other words, according to this embodiment, it is provided that, regardless of the current filling state of the corresponding container, this container is approached by the waste disposal vehicle because a minimum amount of gas has leaked out.

According to another embodiment, the level sensor is attached to the container. The container is, for example, a waste bin. In principle, however, the term “container” is to be interpreted broadly in the context of the disclosure. Likewise, the terms “level sensor” and “gas sensor” as well as “computing unit” are to be interpreted broadly.

According to a further embodiment, the level sensor is a self-sufficient sensor with a battery or the like, which has no wired external power supply. Alternatively or additionally, the gas sensor can be energy-autonomous.

According to a further embodiment, all components of the level sensor are arranged in a plastic housing, possibly also the gas sensor.

According to another embodiment, the plastic housing cannot be opened non-destructively. It is therefore a level sensor that is replaced after the energy storage device has been emptied.

According to another aspect of the present disclosure, the use of a level detection and gas detection system is disclosed, in particular a system described above and below, for planning the route of a waste disposal vehicle. The route could be planned so that bins are emptied according to odor intensity rather than fill level.

It may be envisaged that upon detection of a flammable gas, an installation or parts of an installation are placed in a “protective state”. This can be done by switching off power, equipment or machinery that would be capable of igniting the gas. In addition, containers with flammable substances could be automatically emptied or locked. Likewise, an extinguishing system could be activated preventively before a fire or explosion in order to dilute any spilled substances and thus minimize the risk.

According to another aspect of the present disclosure, the use of a gas sensor for planning the route of a waste disposal vehicle is disclosed.

Another aspect of the present disclosure relates to a method for controlling a level of a container, wherein the level of a medium in the container is sensed and the concentration or presence of a gas and/or an odor in or near the container is sensed or detected. A message, such as a first warning message, is generated and/or a process is instructed when the gas sensor detects a preset gas concentration limit and/or a preset odor intensity limit and/or a predetermined odor. Alternatively or additionally, a message may be generated when the level reaches a preset level limit. A second warning message may then be generated when the gas reaches a preset gas concentration limit.

According to another aspect of the present disclosure, a program element is disclosed which, when executed on a computing unit of a level detection and gas detection system, instructs the level detection and gas detection system to perform the steps described above and below.

Another aspect of the present disclosure relates to a computer-readable medium on which a program element described above is stored.

### BRIEF DESCRIPTION OF THE FIGURES

FIG. 1 shows a level measuring and gas detection system for detecting gases and generating warning messages.

FIG. 2 shows the different concentrations of a gas that are used to locate the point of emission.

FIG. 3 shows a so-called peer-to-peer information flow.

FIG. 4 shows the flow of information via an intermediate system.

FIG. 5 shows a flow chart of a process according to one embodiment.

FIG. 6 shows a level measuring and gas detection system according to a further embodiment.

FIG. 7 shows a graph of odour intensity in relation to odour range.

FIG. 8 shows a schematic communication diagram of a level measuring and gas detection system according to one embodiment.

FIG. 9 shows a flow diagram of a process according to a further embodiment.

### DETAILED DESCRIPTION OF EMBODIMENTS

The illustration in the figures is schematic and not to scale. If the same reference signs are used in the following figure description, these designate the same or similar elements.

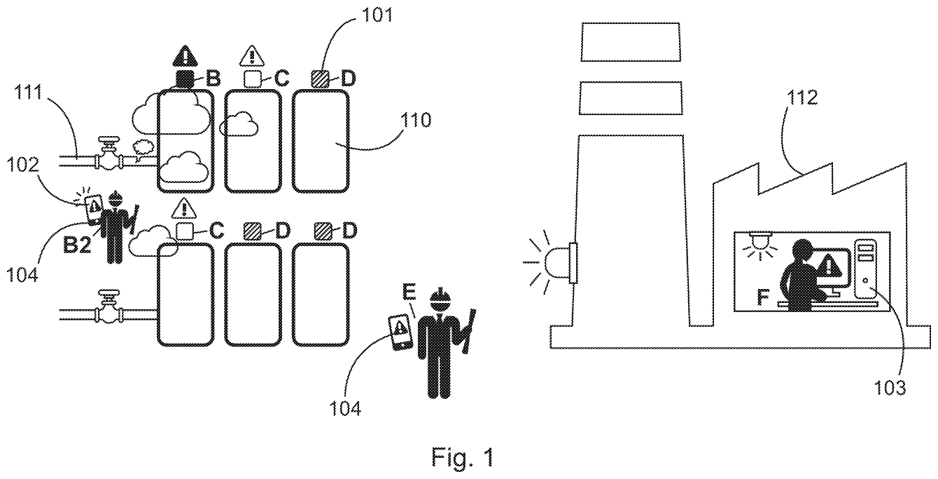

FIG. 1 shows a level measuring and gas detection system 100. The system has a plurality of containers 110, each of which is equipped with a level sensor 101. The first container is connected to a pipe 111 by means of which it can be filled or emptied. Each of the level sensors 101 may be equipped with a gas sensor 102. Also, the system may have a plurality of mobile terminals 104 that may also be equipped with corresponding gas sensors.

Furthermore, a control center 112 is provided, which has a computer unit 103 that collects and evaluates the recorded measurement data of all sensors.

The computing unit 103 can also, as an alternative to a stationary control center, be set up as a cloud or be part of one of the sensors 101, 102.

It is a collective overall system consisting of process sensors (level sensors) and mobile terminals with integrated gas sensors for intelligent detection of increased gas concentrations and for sending out warnings.

By combining several stationary process sensors 101 and mobile terminals 104, such as smartphones, tablets, wearables, with integrated analysis sensors (gas sensors) for detecting gases, the level detection and gas detection system can ensure area-wide detection of escaping gases, which can protect living beings or explosive processes in the environment.

When potential hazards are detected by one or more devices, the overall system can be informed of this hazardous condition, whereupon all connected devices can trigger certain warnings or actions (for example, visual or acoustic warnings or control of further devices and processes).

The communication between the participating devices can be wired or wireless via common radio standards, such as WLAN, Bluetooth, NB-IOT, LoRa, etc. The communication between the participating devices can also be decentralized (peer-to-peer) or centralized (via an intermediate system). The communication between the participating devices can also be decentralized (peer-to-peer) or centralized (via an intermediate system). Such an intermediate system can be used to centrally manage additional tasks, such as system control, archiving of events, access authorizations or alarm points, etc.

The gas sensors can thus be used to generate additional information. The filling level detection and gas detection system can be used in particular in the area of refuse collection and can be set up to detect the information about odor formation in addition to detecting the filling level of refuse bins. For example, it can be provided that in the event of a significant odor nuisance, the route guidance of the refuse vehicle is changed so that, for example, the source of the odor nuisance can be removed as quickly as possible by emptying the corresponding refuse bin.

The gas sensors may be attached to or integrated in the corresponding level sensors. This can also be provided subsequently. Alternatively or in addition to this, mobile operating devices, such as smartphones, can transmit the (gas) additional information to the level sensor(s) or the cloud. Odor data and/or limit values of the odor data can be stored and parameterized in the filling and/or limit level sensor and/or in the cloud to ensure the medium or process quality. For example, corresponding data records can already be stored in the gas sensor and/or in the cloud in the factory if the application is known.

The limit values, or level limit values and/or gas concentration limit values, can be transmitted to any number of level and/or limit sensors. These limit values can be adjusted manually, for example by empirical values.

Since such gas analysis sensors or gas sensors may be designed as complete chip modules, it is possible to equip existing level sensors or mobile operating devices with them. In particular, the gas analysis sensors require little energy, so that it is also possible to equip self-sufficient level sensors with them.

Due to the area-wide detection of potentially hazardous substances in the ambient air, a safety zone can be created depending on the density of the available measuring devices. A significant advantage is that devices that are needed or already in use can be used for this purpose and no additional hardware is required.

The combination of stationary and mobile devices can also ensure that the areas directly around production processes (stationary process sensors), but also around employees (mobile end devices) can be covered.

Another advantage of such an intelligent overall system is the precise geolocation of potential hazards. By combining the measured values of several devices with a known location, overlaps and thus the leakage location of the hazardous substance can be mathematically calculated.

In FIG. 1 you can see how gas escapes at location A. The process sensor B in the upper left tank measures a high gas concentration. Process sensor B2, which is a mobile terminal, also measures a high gas concentration. The two process sensors C, on the other hand, only measure an increased gas concentration, and the three process sensors D do not measure an increased gas concentration. Based on the measurement results, person E is informed about the gas leak via his mobile terminal 104. At the same time, person F sitting in the control center is informed about the gas leak via his stationary device 103.

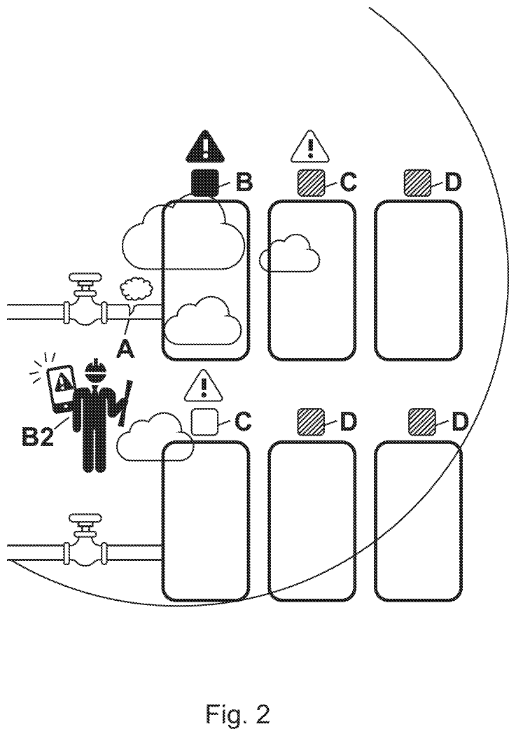

In the situation shown in FIG. 2, a spherical gas concentration distribution decreasing radially outwards can be caused by the leak, which is also shown in FIG. 1. Since the measuring points of devices B and B2 are located at a similar distance from gas leak A, they measure a similarly high gas concentration. Through the measured values of the six measuring devices, which are positioned at different locations, the location of the gas leak can be concluded relatively precisely, since the positions of the six measuring devices are known.

For this purpose, the six units can have position sensors that determine their current position and include it in the calculation.

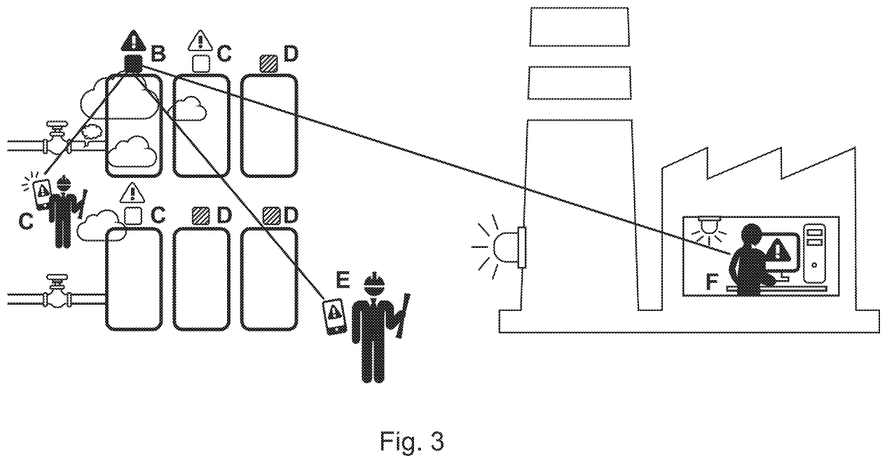

FIG. 3 shows an example of a peer-to-peer information flow in which sensor B, which has measured a very high gas concentration, sends corresponding information directly to the terminal devices involved, in particular terminal devices E and F.

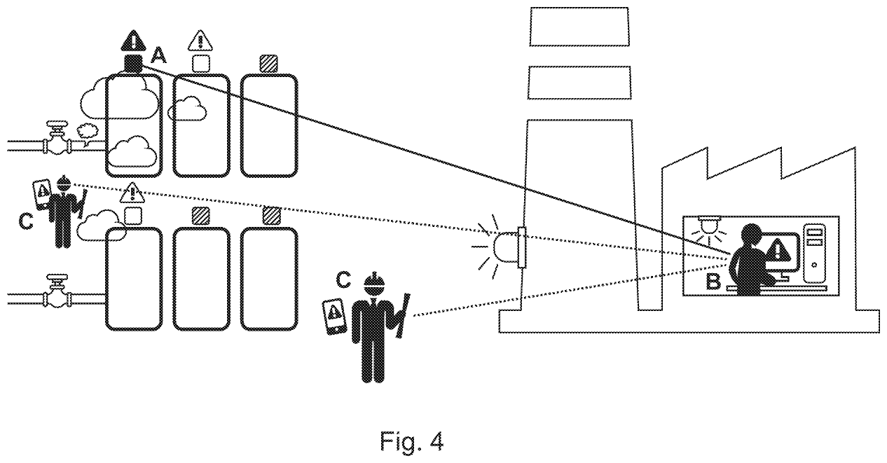

FIG. 4 shows a possible flow of information via an intermediate system, in which the sensor A sends to the intermediate system B (the control center), and then the intermediate system B sends a corresponding warning message to the terminal devices C involved.

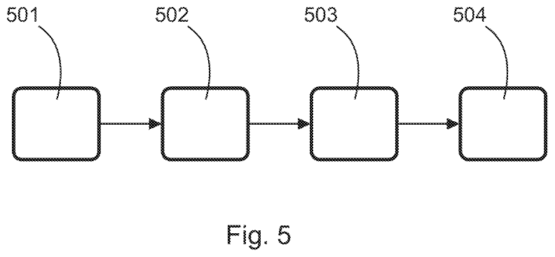

FIG. 5 shows a flow chart of a method according to one embodiment. In step 501, the level of a medium in a container is detected. In step 502, an increased gas concentration is detected in the vicinity of the container and a corresponding warning message is sent in step 503, which then leads in step 504 to the routing of a waste disposal vehicle being changed so that the disturbing odor can be eliminated as quickly as possible.

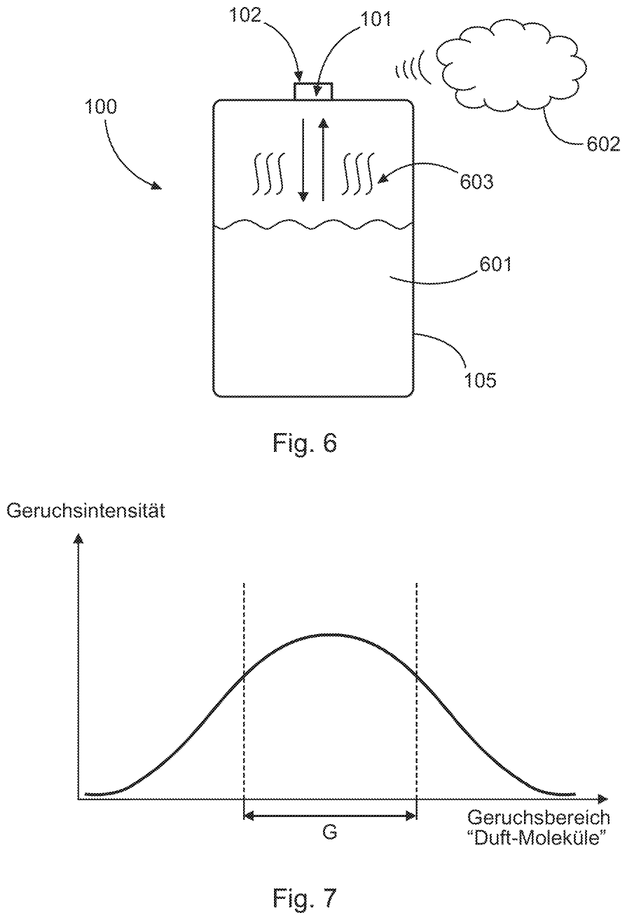

FIG. 6 shows a level measuring and gas detection system according to a further embodiment. The level sensor 101 and the gas sensor 102, or odor sensor, are mounted in a single housing on the container 105. The container 105 is filled with an odorous medium 601. The medium may emit a gas and/or an odor 603. Processing of the sensed data may be performed in the cloud 602. The cloud 602 may comprise the computing unit. Furthermore, the cloud 602 may communicate with the overall level sensing and gas detection system 100. This may eliminate the need for manual odor monitoring by persons. A sensor system in contact with the medium in order to monitor and/or detect the odor may also be omitted. In addition, it is conceivable that an existing sensor, such as a fill level sensor 101, can be retrofitted with a gas sensor 102.

FIG. 7 shows a graph of the odor intensity in relation to the odor range. A predefined permitted odor range G is drawn in the graph.

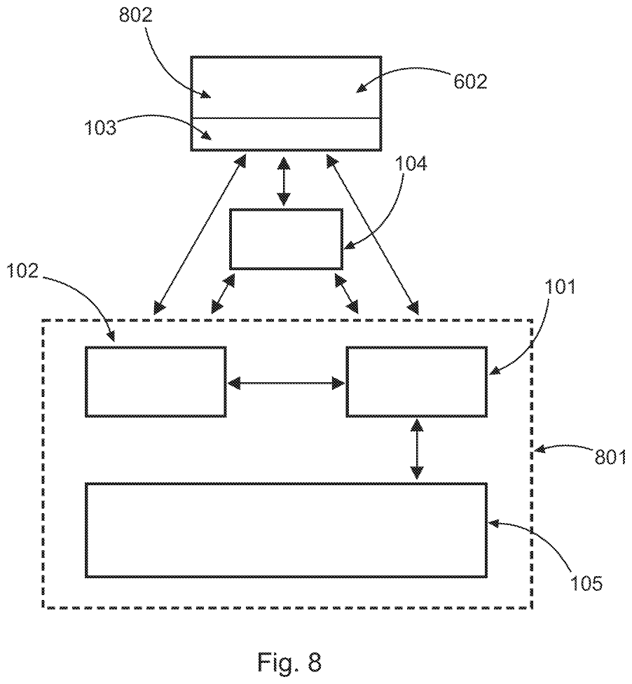

FIG. 8 shows a schematic communication representation of a level detection and gas detection system 100 according to one embodiment. In FIG. 8, the arrows represent a communication, such as a radio communication and/or an interface communication, between the various components, units and/or sensors. The respective communications or communication channels may be two-way communications. For example, the data exchange between the gas sensor 102, or odor sensor, and the level and/or limit sensor 101 may be directly by radio or wired or via the cloud 602. The measuring point 801 of FIG. 8 comprises a container 105, a gas sensor 102 and a level sensor 101. The measuring point 801 can be supplied with data via the gas sensor 102. The computing unit 103 can, for example, be arranged with a memory 802 in a cloud 602.

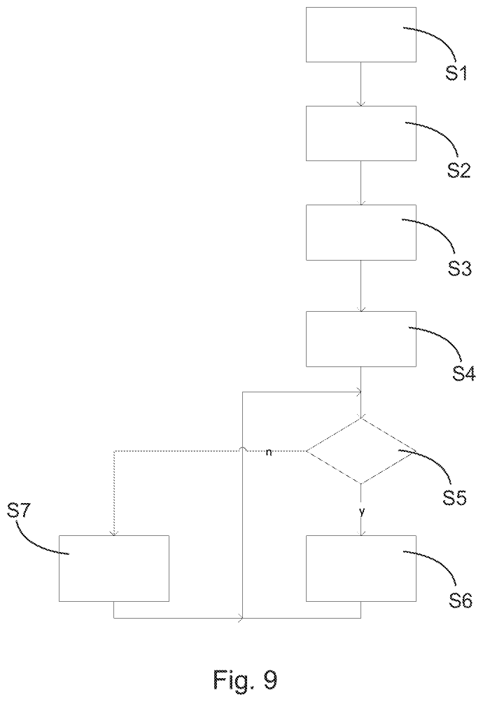

FIG. 9 shows a flow diagram of a method according to a further embodiment. In a first step S1, the gas sensor 102, such as an odor sensor, is calibrated and/or taught. Odor data can then be recorded in a second step S2, for example via a cloud. Limit values can then be set in step S3. In a further step, odor detection can be activated. Thus, an odor intensity or even the presence of an odor at all can be detected or recorded S4. The entire system 100 may perform a verification, such as a confirmation of an allowed odor, in step S5. Based on the results of the check and/or verification of step S5, either the normal operation of the sensor S6 or the system may be instructed, the output of an action, a message and/or an error may be instructed S7 or a re-check may be instructed S5.

In addition, it should be noted that “having” and “comprising” do not exclude other elements or steps and the indefinite articles “an” or “a” do not exclude a plurality. It should also be noted that features or steps described with reference to any of the above embodiments may also be used in combination with other features or steps of other embodiments described above. Reference signs in the claims are not to be regarded as limitations.

1. A level measuring and gas detection system arranged to detect a level and to detect a gas and/or an odor, comprising:
a level sensor configured to measure the level of a medium in a container of a measuring point; and
a gas sensor configured to detect a gas, an odor, and/or an odor intensity;
computing circuitry configured to generate a message and/or to instruct a process when the gas sensor detects a preset gas concentration limit, a preset odor intensity limit, and/or a predetermined odor.

2. The level measuring and gas detection system according to claim 1,
wherein the gas sensor is further configured to determine and store an image of an odor environment of the measuring point.

3. The level measuring and gas detection system according to claim 1, wherein the level measuring and gas detection system is calibrated based on the detected odor.

4. The level measuring and gas detection system according to claim 1,
wherein the odor intensity limit is an odor intensity limit of a permitted or prohibited odor; and/or
where the odor intensity limit is an odor range.

5. The level measuring and gas detection system according to claim 1,
wherein the gas sensor is equipped with nanowires made of tin dioxide.

6. The level measuring and gas detection system according to claim 1,
wherein the gas sensor is integrated in a mobile operating device for the level sensor or is arranged remotely from the level sensor.

7. The level measuring and gas detection system according to claim 1,
wherein the gas sensor is integrated in the level sensor.

8. The level measuring and gas detection system according to claim 1,
wherein the computing circuitry is implemented as a central server or cloud.

9. The level measuring and gas detection system according to claim 1,
wherein the computing circuitry is integrated in the level sensor.

10. The level measuring and gas detection system according to claim 1,
wherein the computing circuitry is further configured to calculate the position of a source of the odor and/or the gas from measurement data detected by the gas sensor.

11. The level measuring and gas detection system according to claim 1,
wherein the process initiated by the computing circuitry is changing a route of a waste disposal vehicle when the gas sensor detects the preset gas concentration limit and/or odor intensity limit.

12. The level measuring and gas detection system according to claim 1,
wherein the level sensor is attached to the container.

13. The level measuring and gas detection system according to claim 1,
wherein the container is a waste container.

14. The level measuring and gas detection system according to claim 1,
wherein the level sensor is a stand-alone sensor that has no wired external power supply and/or draws energy from an energy harvesting system integrated in the level sensor.

15. The level measuring and gas detection system according to claim 1,
wherein all components of the level sensor are arranged in a plastic housing or in a metal housing.

16. The level measuring and gas detection system according to claim 15,
whereby the plastic housing cannot be opened non-destructively.

17. A method of controlling a fill level of a container, comprising:
measuring the fil level of a medium in the container;
detecting a gas and/or an odor in or adjacent to the container;
generating a message, guiding, and/or controlling a process when the gas sensor detects or no longer detects a preset gas concentration limit, a preset odor intensity limit, and/or a predetermined odor.

18. A non-transitory computer readable medium having stored thereon a program element which, when executed on computing circuitry of a level measuring and gas detection system, instructs the level measuring and gas detection system to perform a method comprising:
measuring the level of a medium in a container;
detecting a gas and/or an odor in or near the container;
generating a message, guiding and/or controlling a process when the gas sensor detects or no longer detects a preset gas concentration limit, a preset odor intensity limit, and/or a predetermined odor.
